(19) United States
(12) Patent Application Publication
(10) Pub. No.: US 2024/0013912 A1
(43) Pub. Date: Jan. 11, 2024

(54) COMMUNICATIONS PLATFORM CONNECTING USERS FOR REMOTE MONITORING AND INTERVENTION BASED ON USER-DESIGNATED CONDITIONS

(71) Applicant: EMED LABS, LLC, Miami, FL (US)

(72) Inventor: Nicholas Atkinson Kramer, Wilton Manors, FL (US)

(21) Appl. No.: 18/349,814

(22) Filed: Jul. 10, 2023

Related U.S. Application Data
(60) Provisional application No. 63/367,955, filed on Jul. 8, 2022.
(60) Provisional application No. 63/368,582, filed on Jul. 15, 2022.

(51) Int. Cl. G16H 40/67; G16H 80/00; G16H 50/20

(52) U.S. Cl. CPC G16H 40/67; G16H 80/00; G16H 50/20

(57) ABSTRACT

Systems, methods, and devices associated with telehealth communication platforms are disclosed herein. In some embodiments, a user of the platform may designate a condition that can be used to assign the user to a pool of supervising users. User status data from user tracking devices can be collected and monitored by the platform to detect any abnormalities or indications associated with the user-designated condition, which may indicate a level of urgency and that the user requires an intervention. A communication can then be immediately established between the user and a supervising user selected from the assigned pool based on availability.

### CROSS-REFERENCE TO RELATED APPLICATIONS

This application claims the benefit of U.S. Provisional Patent Application No. 63/367,955, entitled “TRIGGERED THERAPY FOR TIME-SENSITIVE INTERVENTIONS,” filed Jul. 8, 2022, the contents of which are incorporated by reference herein in their entirety. This application also claims the benefit of U.S. Provisional Patent Application No. 63/368,582, entitled “PROCTOR AS HUMAN POINT OF ACCOUNTABILITY,” filed Jul. 15, 2022, the contents of which are incorporated by reference herein in their entirety.

### TECHNICAL FIELD

The embodiments of the disclosure generally relate to technologies for communications platforms. More specifically, the embodiments relate to systems, methods, and devices for telehealth platforms capable of connecting its users in virtual proctoring sessions (e.g., communication over a live audio and/or video stream).

### BACKGROUND

Use of telehealth to deliver healthcare services has grown consistently over the last several decades and has experienced very rapid growth in the last several years. Telehealth can include the distribution of health-related services and information via electronic information and telecommunication technologies. Telehealth can allow for long distance patient and health provider contact, care, advice, reminders, education, intervention, monitoring, and admissions. Often, telehealth can involve the use of a user or patient's personal user device (e.g., a smartphone, tablet laptop, personal computer, or other device). For example, a user or patient can interact with a remotely located medical care provider using live video, audio, or text-based chat through the personal user device. Generally, such communication occurs over a network, such as a cellular or internet network.

However, much of telehealth development has been focused on strictly replicating the interaction between a patient and medical care provider but in a virtual setting (e.g., a real-time, synchronous video conference conducted on a 1:1 basis). Unsurprisingly, this has resulted in many other features of traditional medicine being carried over, such as scheduled appointments and initial consultations with generalists, thereby limiting the overall efficiency, scalability, and potential of telehealth.

Accordingly, there exists a need for configuring telehealth platforms to provide better service and improved patient outcomes, in a way that is impossible for traditional medical paradigms. For example, telehealth presents the opportunity for providing users with continuous monitoring and detection or access to always-available, immediate responses from trained professionals. Furthermore, telehealth could also leverage digital tools and connectivity to motivate and improve patient adherence to a medical treatment plan or regimen.

### SUMMARY

For purposes of this summary, certain aspects, advantages, and novel features are described herein. It is to be understood that not necessarily all such advantages may be achieved in accordance with any particular embodiment. Thus, for example, those skilled in the art will recognize the disclosures herein may be embodied or carried out in a manner that achieves one or more advantages taught herein without necessarily achieving other advantages as may be taught or suggested herein.

All of the embodiments described herein are intended to be within the scope of the present disclosure. These and other embodiments will be readily apparent to those skilled in the art from the following detailed description, having reference to the attached figures. The invention is not intended to be limited to any particular disclosed embodiment or embodiments.

The embodiments of the disclosure generally relate to systems, methods, and devices for connecting users of telehealth platforms that offer virtual proctoring (e.g., communication over a live audio and/or video stream).

In some embodiments, a user (e.g., a patient) of a telehealth proctoring platform may designate a condition (e.g., an ailment or medical condition), which can be used to assign the user to a pool of supervising users (e.g., proctors) that are experienced and trained in handling and providing intervention on that condition. User status data can also be transmitted from user tracking devices (e.g., devices with health sensors or health monitors) and collected by the telehealth proctoring platform. The telehealth proctoring platform can process and monitor the user status data to detect any abnormalities or indications associated with the user-designated condition, which may indicate a level of urgency and that the user requires an intervention. A communication (e.g., a virtual proctoring session) can then be immediately established between the user and a supervising user (e.g., proctor) selected from the assigned pool based on availability. The communication can be used to provide intervention to the user that has a high likelihood of relevancy and efficacy due to the experience that the supervising user will have of the user-designated condition. Thus, the telehealth proctoring platform can provide better service and improved patient outcomes, in a way that is impossible for traditional medical paradigms. More specifically, the platform can provide its users with continuous monitoring and detection or access to always-available, immediate responses from trained professionals (e.g., by connecting the user to a professional based on the specific user condition that is at issue).

In some embodiments, a user (e.g., a patient) of a telehealth proctoring platform may be enrolled in a medical treatment plan or health improvement regimen managed over the platform. The telehealth proctoring platform may allow for various motivational programs or components to be assigned to the user in order to motivate continued adherence to the treatment plan. These programs can be assigned by medical professionals, such as by doctors to high-risk patients for whom adherence to a treatment plan is critical. The programs may also be started voluntarily on an opt-in basis by users. These motivational programs may be administered by a supervising user (e.g., a proctor), and they may implement communications (e.g., virtual proctoring sessions) between the user and the supervising user in various ways to aid in completion of the health improvement regimen. For instance, the user may have to communicate daily with the supervising user to confirm that certain tasks specified by the regimen have been completed. The supervising user can use the information obtained from the motivational program for troubleshooting purposes and to modify the treatment plan for better adherence. In some embodiments, the supervising user administering the motivational program to the user may be pulled from a group of idle supervising users (e.g., idle proctors) who also administer other telehealth services over the platform, which can help balance resource utilization curves and the burden put on supervising users. Thus, the telehealth proctoring platform can provide better service and improved patient outcomes, in a way that is impossible for traditional medical paradigms. More specifically, the platform can leverage digital tools and connectivity to motivate and improve patient adherence to a medical treatment plan or regimen.

Accordingly, the different embodiments disclosed herein may contemplate various approaches for at least: collecting, processing and monitoring user status data transmitted from user tracking devices to determine appropriate interventions to take; connecting different users for remote monitoring and intervention based on user-designated conditions; using digital tools and connectivity to motivate users to adhere to treatment plans or to adjust treatment plans; and enabling the remote monitoring, intervention, motivation, and troubleshooting of users of virtually proctored telehealth platforms. Among other things, these embodiments may improve platform efficiency and scalability, improve intervention speed and quality, and also improve user adherence (e.g., to a treatment plan or regimen).

### BRIEF DESCRIPTION OF THE DRAWINGS

These and other features, aspects, and advantages of the disclosure are described with reference to drawings of certain embodiments, which are intended to illustrate, but not to limit, the present disclosure. It is to be understood that the accompanying drawings, which are incorporated in and constitute a part of this specification, are for the purpose of illustrating concepts disclosed herein and may not be to scale.

FIG. 1 illustrates a system diagram of a telehealth proctoring platform that can be used for remote proctoring, monitoring, intervention, motivation, and troubleshooting of its users, in accordance with embodiments disclosed herein.

FIGS. 2A-2B illustrate block diagrams of example methods that can be used to provide faster and more specific monitoring, detection, and intervention for users of a telehealth proctoring platform, in accordance with embodiments disclosed herein.

FIGS. 3A-3C illustrate block diagrams of methods associated with different example motivational programs that can be assigned to a patient enrolled in a medical treatment plan managed over a telehealth proctoring platform, in accordance with embodiments disclosed herein.

FIG. 4 presents a block diagram illustrating an embodiment of a computer hardware system configured to run software for implementing one or more embodiments of the systems and methods disclosed herein.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

In general, traditional medicine depends on scheduled appointments at physically distant locations. This poses a problem for medical issues that require time-sensitive interventions. Even emergency services like paramedics require travel time and going to a physical hospital. Telemedicine provides an opportunity for always-available immediate responses from trained professionals in a way that is impossible for traditional medical paradigms.

In some embodiments, patients with designated ailments can be attached to a pool of telemedicine practitioners who are trained to help with a specific time-sensitive condition. For example, patients prone to anxiety attacks can be rapidly or even automatically connected to a practitioner that walks them through grounding exercises such as reciting facts about where they are, breathing exercises, or even acute therapy sessions. These can be augmented by in-app or on-website visuals or instructions-for-use (“IFUs”) to go along with the intervention exercises.

Due to the increasing prevalence of wearable technology, one can use one or more pieces of data, such as bioindicators from health tracker sensors, GPS or IMUs to detect these time-sensitive ailments perhaps even before the user is aware of them. For example, a patient with hypertension whose blood pressure spiked may be automatically connected with a proctor who has them perform a somatic meditation while also assessing if they need to go to the hospital for a heart attack risk. As another example, a patient with anxiety attacks might have elevated BPM and hyperoxygenation of the blood detected along with a GPS location indicating they are near one of their triggers (e.g., large crowds, a workplace, etc.), and so may be contacted before the patient realizes they are about to have an attack to help guide them through it or even prevent it.

For some interventions, it may be desirable to provide minimal intervention with optional escalation of care. For example, a system, such as an artificial intelligence system, con be configured to provide initial communication with the patient. For example, through an application or other method, the system can, for example, say “Hey, would you like to take a break and do a quick breathing exercise with me?” The system can further ask, “If you′d like to speak to one of our specialists, at any time please ask for assistance. But in the meantime, let's go through a somatic meditation checklist.” Depending on the outcome of the checklist, the patient interaction can be escalated to a trained telemedicine specialist as needed or even to an ambulance being dispatched.

Some patients may even authorize an automatic activation of sensors on their devices for medical practitioners or telemedicine system or platform to make rapid judgment calls. For example, a recovered drug addict may be wearing an Apple Watch, FitBit, or other health tracker that gives a worrying indication. In response, a telemedicine practitioner could pull up (without user prompting) a data panel on the patient that can include, for example, biomarkers such as blood oxygen levels, a graph of heart rate, a video feed from either/both device (e.g., smartphone) cameras, a microphone feed, an ability to speak out of the speakers, a three dimensional model of the patient moving around the room using IMU data, along with estimated patient orientation (laying on ground, sitting), limb movement via smartwatch IMU, etc. Based on this information, the telemedicine practitioner, system, or platform could then make a judgment call to disconnect, to stay on the line and monitor, to start engaging with the patient, or to dispatch an ambulance.

Patient adherence to a health improvement regimen such as drug abstinence, medicine or prescription schedules, diet, exercise, and/or any other health improvement or lifestyle change. Patient adherence is a leading indicator of whether the health improvement regimen will be successful in improving patient health. For long adherence to the health improvement regimen, the first 60-90 days are critical. In this time period, patients are the most likely to break a habit and make excuses as to why the patient skipped a day or broke the habit. Typically, these excuses fall apart under scrutiny, however, without the scrutiny, patients can convince themselves the excuse is a legitimate excuse. Therefore, to ensure the success of the health improvement regimen when adherence to the health improvement regimen is critical for a high-risk patient, external accountability can be provided via telehealth.

In some embodiments the systems, methods, and devices may include one or more motivational components. The one or more motivational components can be delivered via a proctored telemedicine or telehealth platform. In some embodiments, a doctor can assign the one ore motivational components to a patient. The patient can be a high-risk patient. In some embodiments the patient can voluntarily opt-in to or sign up for the one or more motivational components for self-improvement, habit changes, and/or other lifestyle changes. In some embodiments, a telemedicine proctor or a telemedicine practitioner can administer the one or more motivational components. The telemedicine proctor or the telemedicine practitioner can administer other telemedicine or telehealth services, such as COVID-19 testing, flu testing, UTI testing, VpH testing, HPV testing, and/or any other telemedicine or telehealth services. The system can pull or use proctors from a group of idle or unused proctors who administer the other telemedicine or telehealth services. In some embodiments, the one or more motivational components can be a part of a same telemedicine platform used for the other telemedicine or telehealth services. In some embodiments, the one or more motivational component can be a separate telemedicine platform.

In some embodiments, the patient can periodically meet with the telemedicine proctor or telemedicine practitioner via telehealth. In some embodiments, the patient can meet with the telemedicine proctor or practitioner daily. In other embodiments, the patient can meet with the telemedicine proctor or practitioner weekly. In some embodiments, the meeting can take place in the morning or the evening. The patient can make a verbal commitment to the proctor.

The verbal commitment can include one or more actions the patient will perform. Performing the action can include abstaining from an action. In some embodiments, the verbal commitment can include when and/or how the action will be performed. In some embodiments, the patient can check-in with the telemedicine proctor or telemedicine practitioner via an app, a phone call, messaging and or any other form of communication. The form of communication can depend on patient preferences and/or user studies. In some embodiments, when the patient makes the verbal commitment, the commitment is externalized, and the commitment can be paced into a mental calendar of the patient. In some embodiments, externalizing the commitment can increase a likelihood of success.

In some embodiments, after completing a task, the patient can connect to the telemedicine proctor or telemedicine practitioner to report to completion of the health improvement regimen or a portion thereof. Additionally, the patient can provide information relevant to the completion of the health improvement regimen. For example, the patient could say it was hard to abstain today, I 660 calories, I took my full dose, or I completed 5×5 80-pound squats. The telemedicine proctor or telemedicine practitioner can enter the information into a file. In some embodiments, the telemedicine proctor or telemedicine practitioner can provide feedback to the patient. The feedback can include the telemedicine proctor or telemedicine practitioner congratulating the patient and/or displaying a congratulatory animation on a display of a patient device. In some embodiments, if the patient does not complete a portion of the health improvement regimen, the telemedicine proctor or telemedicine practitioner can encourage the patient and/or assist with troubleshooting or overcoming obstacles. In some embodiments, the telemedicine proctor or telemedicine practitioner and/or the patient can update the health improvement regimen. In some embodiments, updating the health improvement can include the patient meeting with a prescriber to update one or more medications. The patient can meet with the prescriber via an app, a call, a video conference, a website, or an in-person visit. In some embodiments, telemedicine proctor or telemedicine practitioner can receive a notification that the patient cannot complete at least a portion of the health improvement regimen.

In some embodiment, the patient can be automatically connected with telemedicine proctor or telemedicine practitioner if the patient does not check in by a predetermined time of day. In some embodiments, the patient can explain to the telemedicine proctor or telemedicine practitioner why the patient did not complete at least a portion of the health improvement regimen. The patient may have a difficult time justifying excuses to the telemedicine proctor or telemedicine practitioner, which may increase a mental burden of not completing at least a portion of the health improvement regimen and a mental reward of completing at least a portion of the health improvement regimen.

In some embodiments, the check in can provide a reminder or reinforcement to the patient that the patient completed the portion of the health improvement regimen. The reminder or reinforcement can improve confidence, habit formation, and/or peace of mind.

In some embodiments, the system can provide one or more visuals or animations as the reminder or reinforcement. The system can provide the patient with a digital calendar. The digital calendar can identify or stylize completed tasks and uncompleted tasks.

In some embodiments, the system can create one or more artifacts or records. In some embodiments, only the telemedicine proctor or telemedicine practitioner can input data or information into the artifact or record. The patient can verbally provide the data or information to the telemedicine proctor or telemedicine practitioner. In some embodiments, the patient can have more difficulty providing false data or information to the telemedicine proctor or telemedicine practitioner than the patient would if the patient entered the information themselves. In some embodiments, the telemedicine proctor or telemedicine practitioner can be trained to ask pressing information. In some embodiments, the data or information can depend on the health improvement regimen of the patient.

In some embodiments, the one or more artifacts can include a certificate for completing one or more portions of the health improvement regimen, a display of success as a graph, a physical object, a figurine, a trophy, and/or a graphic that the patient can share via email, social media or any other communication method. The one or more artifacts can be a real-world tangible representation of completion of one or more portions of the health improvement regimen, and the one or more artifacts can provide accountability and motivation.

In some embodiments, the system can automatically and dynamically generate records or reports about progress of the patient. The record or reports can be sent to the patient's healthcare provider. In some embodiments, the record or reports can be provided to the healthcare provider upon request, at regular intervals, when a patient misses the check in, and/or upon completion of the health improvement regimen.

In some embodiments, the system can group a plurality of patients together. The patients can be assigned the same health improvement regimen. The group can provide each patient accountability from other patients experiencing similar challenges. The plurality of patients can provide suggestions for how to deal with challenges, and/or support to each other.

In some embodiments, the health improvement regimen can be linked with one or more devices or events in the patient's day. In some embodiments, an alarm clock may trigger a timer. The timer can indicate what time the patient's check in is at. In some embodiments, the system can access a location of a device of the patient. When the location of the device indicates the patient is home from work, school, or any other task, the system can trigger the timer.

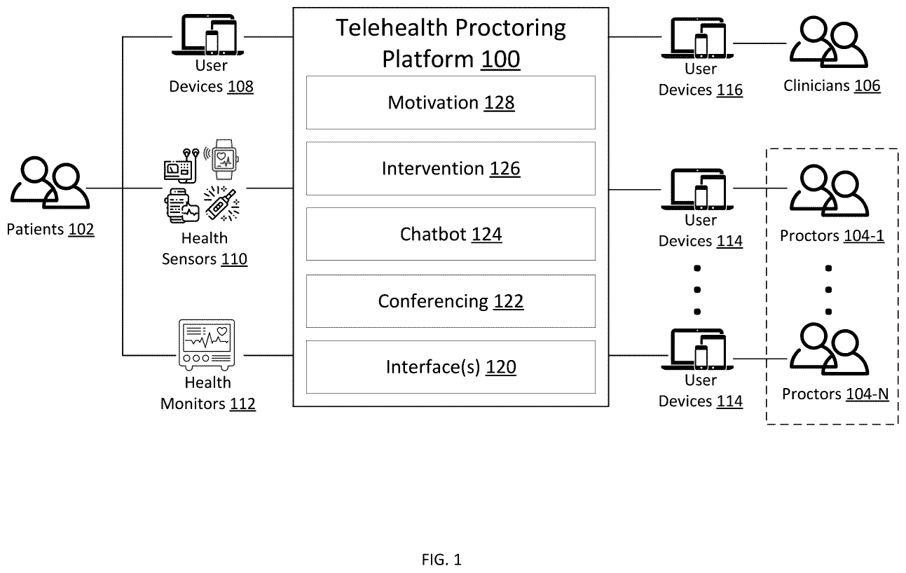

FIG. 1 illustrates a system diagram of a telehealth proctoring platform that can be used for remote proctoring, monitoring, intervention, motivation, and troubleshooting of its users (e.g., patients of telehealth services), in accordance with embodiments disclosed herein. More specifically, it illustrates a system diagram of a telehealth proctoring platform 100.

It should be noted that functionality of the telehealth proctoring platform 100 may be described and/or shown as components, modules, and systems. These illustrations and descriptions of the various components of the telehealth proctoring platform 100 are provided for the purpose of facilitating ease of understanding. In practice, the functionality of the telehealth proctoring platform 100 does not need to be siloed or delineated in the same manner. For instance, one or more of the components may be optional, used together, or combined. Furthermore, one or more of the components may be separately located (e.g., with a third-party or at an endpoint) or their corresponding functionality may be performed at various locations. For example, the interfaces 120 may include user interfaces displayed to a patient through a website or web-based application (with the user interface data provided by a server on the backend), or alternatively, the user interfaces could be displayed by an application running on the patient's user device—e.g., user device 108 (and that application may or may not be part of the telehealth proctoring platform 100).

It also should be noted that the term “users” may sometimes refer to the patients 102 (who are proactively using the telehealth proctoring platform 100), especially in the context of self-administering medical diagnostic tests, participating in a medical treatment plan, or availing themselves of telehealth services. However, the term “users” may also refer to any of the patients 102, proctors 104, and/or clinicians 106 (who could all be considered users in the literal sense since they all interact with the telehealth proctoring platform 100). The term “supervising users” may refer to the proctors 104 and/or clinicians 106, who may be able to supervise patients 102 as they self-administer diagnostic tests and medication, inquire about ailments/conditions that they may be experiencing, perform tasks in accordance with a medical treatment plan, and so forth. In some embodiments, the term “supervising users” may also refer to an AI chatbot or system that interacts with and provides guidance to the patients 102 in a humanlike manner despite not actually being human.

In some embodiments, the patients 102, proctors 104, and/or clinicians 106 may be able to interact with the telehealth proctoring platform 100 through one or more interfaces 120 associated with the telehealth proctoring platform 100. These interfaces 120 may include various user interfaces and/or application programming interfaces (APIs) depending on the implementation of the telehealth proctoring platform 100. For example, in some embodiments, one or more of the patients 102, proctors 104, and/or clinicians 106 may access the telehealth proctoring platform 100 via their user device (e.g., the user devices 108, 114, and 116) by accessing an application installed on their user device or a web-based application, which will provide various user interfaces that can be interact with. In some embodiments, one or more of the patients 102, proctors 104, and clinicians 106 may access an application installed on their user device or a web-based application, and that application may communicate with the telehealth proctoring platform 100 via an API.

Generally, the patients 102 may include any person that has availed themselves of telehealth services via the telehealth proctoring platform 100. The patients 102 may include any person self-administering a medical diagnostic test (e.g., a lateral flow test used to detect the presence of an analyte in a sample) or self-administering medication (e.g., an injectable drug). The telehealth proctoring platform 100 can be used to provide virtual proctoring and supervision to ensure that those procedures are performed properly and authentically. The patients 102 may include any person receiving or registered to receive medical treatment as part of a medical treatment plan, such as a weight loss program. The telehealth proctoring platform 100 can be used to evaluate compliance and adherence to the medical treatment plan and to provide motivation and encouragement. The patients 102 may include any persons with known ailments or proclivities towards developing certain medical conditions. The telehealth proctoring platform 100 can be used to allow telemedicine practitioners (e.g., proctors 104 and/or clinicians 106) specifically possessing experience and training on those ailments and conditions to be assigned to monitor the person and immediately intervene when the person needs assistance.

The patients 102 may utilize various user devices 108 to connect to the telehealth proctoring platform 100 and attend virtual proctoring sessions. Non-limiting examples of the user devices 108 (and also the user devices 114 and 116) may include a mobile or other handheld device (e.g., cell phone, smartphone, tablet, laptop, etc.). In some embodiments, the patients 102 may be able to interact with the telehealth proctoring platform 100 through one or more interfaces 120 associated with the telehealth proctoring platform 100. These interfaces 120 may include various user interfaces and/or application programming interfaces (APIs) depending on the implementation of the telehealth proctoring platform 100. For example, in some embodiments, the patients 102 may have direct access to the telehealth proctoring platform 100 via the user devices 108 (e.g., via an installed application, web-based application, website, etc.), which can display user interfaces that the patients 102 can interact with.

The patients 102 may have user tracking devices that continuously monitor and collect user status data (e.g., any kind of data associated with the user, such as GPS location data). In some cases, the user devices 108 may be user tracking devices (e.g., a smartphone may track the GPS location of the patient 102). In some cases, the user tracking devices may be able to directly send the data to the telehealth proctoring platform 100. In some cases, the user tracking devices may be able to indirectly send the data to the telehealth proctoring platform 100, such as by providing the data to the user devices 108 for transmission to the telehealth proctoring platform 100.

These user tracking devices may include health tracking devices, such as health sensors 110 and/or health monitors 112, that continuously monitor and collect the patient's real-time health data (e.g., health information, bioindicators, physiological data, vitals, etc.). For the purposes of facilitating understanding of the application, the collection, transmission, and processing of data from health tracking devices (such as health sensors 110 and/or health monitors 112) is of primary focus and discussion, but it should be understood that data from any suitable user tracking device may be similarly transmitted and utilized by the telehealth proctoring platform 100.

Some non-limiting examples of health sensors 110 and/or health monitors 112 may include remote patient monitoring (RPM) devices, point-of-care sensing devices, and wearable technology such as smart watches and health/fitness trackers. For instance, wearable technology is often used to monitor a user's health. Wearables can be used to collect data on a user's health including: heart rate, calories burned, steps walked, blood pressure, release of certain biochemicals, time spent exercising, seizures, physical strain, body composition, and water levels. Additionally, wearable technology may be used to monitor glucose, alcohol, lactate, blood oxygen, breath monitoring, heartbeat, heart rate and its variability, electromyography (EMG), electrocardiogram (ECG) and electroencephalogram (EEG), body temperature, pressure (e.g. in shoes), sweat rate or sweat loss, and levels of uric acid and ions—e.g. for preventing fatigue or injuries or for optimizing training patterns. Additionally, wearable technology may be used to measure mood, stress, and health; measure blood alcohol content; measure athletic performance; monitoring how sick the user is; detect early signs of infection; perform long-term monitoring of patients with heart and circulatory problems by recording an electrocardiogram; perform days-long continuous imaging of diverse organs via a wearable bioadhesive stretchable high-resolution ultrasound imaging patch (e.g. a wearable continuous heart ultrasound imager); perform sleep tracking, monitor cortisol levels for measuring stress; and measure relaxation or alertness, e.g. to adjust their modulation or to measure efficacy of modulation techniques.

The proctors 104 may include medical professionals (e.g., physician, nurse, nutritionist, health coach, and/or the like) that can monitor, supervise, and provide instructions or real-time guidance to the patients 102 (e.g., in a virtual proctoring session facilitated by the telehealth proctoring platform 100). The proctors 104 may be able to perform various roles for many different situations and contexts. For example, a proctor 104 may be able to supervise a patient 102 performing a medical diagnostic test to verify adherence to proper test procedure. The proctor 104 may be able to ensure test result authenticity (e.g. that the test results have not been swapped or tampered with) or even provide suggestions or interpretations of the diagnostic test results. More specifically, a proctor 104 could virtually meet with a patient 102 to go over instructions for a lateral flow test to detect COVID-19 and then assist the patient 102 with interpreting the results of the lateral flow test. In another similar example, the proctor 104 may be able to supervise a patient 102 while they self-administer a medication (e.g., inject themselves with a drug) to provide instructions and guidance on how to administer the medication correctly (e.g., the exact location the drug should be injected, the correct dosage, and so forth).

In some embodiments, a proctor 104 may be able to virtually meet with a patient 102 to go over instructions for a medical treatment plan (e.g., a weight loss program), to monitor the patient's progress during the medical treatment plan, and to confirm the patient is adhering to the plan. In some embodiments, the proctor 104 may be able to assign a motivational program or component to a patient 102 enrolled in a medical treatment plan. In some embodiments, the proctor 104 may be able to administer the motivational program to the patient 102, the process for which may vary depending on the motivational program. For instance, the proctor 104 may hold a daily communication with the patient 102 to receive updates from the patient 102 about completion of tasks specified by the treatment plan; the proctor 104 may provide congratulation, feedback, or encouragement to the patient 102; and the proctor 104 may also help the patient 102 troubleshoot any obstacles that may have prevented full completion of the plan or help the patient 102 modify/adjust the plan so that it can be fully completed going forward.

In some embodiments, the proctors 104 may be trained or experienced in handling varying ailments or medical conditions. A patient 102 having a known ailment or medical condition can be assigned one or more proctors 104 that specifically possess experience and training for it. Those assigned proctors 104 may be able to monitor that patient 102 to detect symptoms of, or the worsening of, the ailment/condition. In some cases, those proctors 104 may be able to monitor the patient 102 through virtual proctoring sessions (e.g., live video streams, telehealth conferences, etc.). In some cases, those proctors 104 may be able to monitor the patient 102 asynchronously (e.g., without the patient 102 initiating a virtual proctoring session), such as by evaluating health information, bioindicators, physiological data, vitals, or other quantitative data associated with the patient 102. For example, the patient 102 may have health sensors 110 and/or health monitors 112 that continuously collect the patient's health information to send to the telehealth proctoring platform 100. In turn, that data may be presented in a visually intuitive and easily understood manner to any proctors 104 that are assigned to that patient 102 (e.g., a recent or real-time ECG graph for a patient undergoing a heart attack). Based on their training and experience, those proctors 104 may be able to easily detect when the data indicates something is wrong and they can immediately intervene when the patient needs assistance (e.g., call an ambulance, provide the patient instructions/guidance, etc.).

Accordingly, in some embodiments, the patients 102 having a designated ailment/condition can be attached to a corresponding pool of telemedicine practitioners (e.g., proctors 104 and/or clinicians 106) that are trained to assist with that particular ailment/condition. As shown in the figure, the proctors 104 are shown to be allocated into a set of N pools (e.g., starting from proctors 104-1 to proctors 104-N) for different ailments/conditions. In some cases, there may be overlap among the pools (e.g., a particular proctor may be associated with multiple pools); a proctor may be assigned to a pool based on their training and experience with the ailment/condition associated with that pool, and there may be proctors that have training and experience to handle many different ailments and conditions.

Thus, a patient 102 that is suffering from, or needs assistance with, an ailment/condition may be connected to an available proctor (e.g., via a virtual proctoring session) that is selected from a pool of proctors that is associated with that specific ailment/condition. In some cases, the ailment or condition may be time-sensitive (e.g., the patient may need to speak with someone right away) and having a pool of proctors to draw from may allow for on-demand virtual proctoring sessions that can be immediately conducted instead of having to be scheduled well in advance. For example, patients 102 that are prone to anxiety attacks can be rapidly or even automatically connected to a proctor selected from a pool of proctors that handle anxiety attacks (e.g., proctors 104-1). These proctors may be trained to mitigate anxiety attacks through intervention exercises; they can walk the patients 102 through grounding exercises (such as reciting facts about where they are), breathing exercises, or even acute therapy sessions. In some embodiments, these virtual proctoring sessions can be augmented by in-app or on-website visuals or instructions-for-use (“IFUs”) to go along with the intervention exercises.

The proctors 104 may utilize various user devices 114 to connect to the telehealth proctoring platform 100 and perform telemedicine functions. In some embodiments, the proctors 104 may be able to interact with the telehealth proctoring platform 100 through one or more interfaces 120 associated with the telehealth proctoring platform 100. These interfaces 120 may include various user interfaces and/or application programming interfaces (APIs) depending on the implementation of the telehealth proctoring platform 100. For example, in some embodiments, the proctors 104 may have direct access to the telehealth proctoring platform 100 via the user devices 114 (e.g., via an installed application, web-based application, website, etc.), which can display user interfaces that the proctors 104 can interact with to perform telemedicine functions (e.g., attend a virtual proctoring session).

The clinicians 106 may refer to any doctor that has contact with, and direct responsibility for, a patient and is capable of approving or modifying a patient's medical treatment plan. There may be some overlap between the roles/functions of the proctors 104 and the clinicians 106 (e.g., both may be considered telemedicine practitioners and engage in telehealth conferencing with patients). In some cases, there may not be much distinction between the two or the distinction may be difficult to discern. However, in some cases, the clinicians 106 may be able to make additional decisions that many of the proctors 104 would not be able to make, such as modifications to a patient's medical treatment plan or prescribed medication.

The clinicians 106 may utilize various user devices 116 to connect to the telehealth proctoring platform 100 and perform telemedicine functions. In some embodiments, the clinicians 106 may be able to interact with the telehealth proctoring platform 100 through one or more interfaces 120 associated with the telehealth proctoring platform 100. These interfaces 120 may include various user interfaces and/or application programming interfaces (APIs) depending on the implementation of the telehealth proctoring platform 100. For example, in some embodiments, the clinicians 106 may have direct access to the telehealth proctoring platform 100 via the user devices 116 (e.g., via an installed application, web-based application, website, etc.), which can display user interfaces that the clinicians 106 can interact with to perform telemedicine functions (e.g., attend a virtual proctoring session, review a patient's health records, make modifications to a prescribed medication or medical treatment plan, etc.).

In some embodiments, the telehealth proctoring platform 100 can include a conferencing system or module 122. In some embodiments, the conferencing module 122 can be configured to connect a patient 102 and a proctor 104 in a telehealth or virtual proctoring session. In some embodiments, the conferencing module 122 can be configured to connect the patient 102 and the proctor 104 via a video conferencing session, such as via live video (e.g., over the Internet of cellular communication network). In some embodiments, the conferencing module 122 can be configured to facilitate video calls, audio calls and/or telemedicine calls. In some embodiments, the patient 102 may access the conferencing module 122 via their user device 108, and the proctor 104 may access the conferencing module 122 via their user device 114 (e.g., a proctor device).

In some embodiments, the conferencing module 122 may be configured to establish a live, virtual proctoring session between a patient and a proctor. For example, it may enable a patient 102 to provide to a proctor 104 a live video feed of the patient 102 (e.g., performing a diagnostic test). In some cases, a patient may be assigned to a specific proctor or a group of proctors in advance (e.g., to a particular medical professional or group of medical professionals). In some cases, the patient may be assigned to one of the proctors 104 based on availability (e.g., who is available when the patient initiates a proctoring session), and/or based on personal considerations (e.g., the patient's sex, gender, age, co-morbidities, dietary preferences, and so forth). In some cases, a patient having a designated ailment/condition can be attached to a corresponding pool of proctors that are trained to assist with that ailment/condition; a patient with multiple ailment/conditions may be attached to multiple corresponding pools. A patient that is suffering from, or needs assistance with, a particular ailment/condition may be connected to an available proctor that is selected from the pool of proctors associated with that ailment/condition.

Virtual proctoring sessions may be scheduled (e.g., at regular intervals as part of a medical treatment plan) or provided on-demand (e.g., to monitor performance of a diagnostic test, to confer with a patient needing immediate assistance with an ailment/condition, etc). In some cases, a patient may be the initiator of scheduled or on-demand virtual proctoring sessions. In other cases, the telehealth proctoring platform 100 itself or a proctor 104 may be the initiator of scheduled or on-demand virtual proctoring sessions. For example, a schedule of regular and periodic check-ins for a medical treatment plan can be established for the patient. As another example, a proctor 104 evaluating the patient's health information may catch troubling indications (e.g., high blood pressure) associated with the patient's ailment/condition and initiate a virtual proctoring session to bring it to the patient's attention.

Thus, the flexibility (e.g., remote, scheduled or on-demand, and seamlessly managed by the telehealth proctoring platform 100) of the virtual proctoring sessions and the manner that they are implemented in the telehealth proctoring platform 100 may provide numerous benefits, and a non-inclusive list is provided. First, they allow the telehealth proctoring platform 100 to enable greater interaction between the patient and a medical professional. The virtual proctoring sessions can be used to easily track patient compliance with a medical treatment plan or testing procedure by monitoring or tracking a patient's self-administration of a procedure, treatment, or medication. Not only can the medical professional give feedback and guidance, but the medical professional can also easily provide additional motivation (e.g., to inspire continued patient compliance and adherence with a medical treatment plan). Second, the virtual proctoring sessions allow for a patient to connect with medical professionals having experience and training specific to their particular ailment/condition. This means that any guidance or intervention provided to the patient will be more effective and better-suited for that ailment/condition.

In some embodiments, the telehealth proctoring platform 100 can include a chatbot system or module 124. The chatbot module 124 may be an artificial intelligence (AI) system capable of maintaining a conversation with a user in natural language and simulating the way a human would behave as a conversational partner. Such technologies often utilize aspects of deep learning and natural language processing. In some embodiments, the chatbot module 124 can be configured to provide initial communication and interaction with a patient, such as a patient dealing with a particular ailment/condition. In some embodiments, the chatbot module 124 may be designed or customized to further target even more specific situations and/or particular subject-matter domains. For example, the chatbot module 124 may be configured to be knowledgeable about the various different kinds of interventions for different ailments/conditions, and it may be able to directly perform some of those interventions (e.g., walk a patient through the steps of a breathing exercise).

In some cases (e.g., for certain ailments/conditions and interventions), it may be desirable to initially provide minimal intervention with optional escalation of care, and the chatbot module 124 may be able to fulfill this role. For example, for a patient having an anxiety attack, the chatbot module 124 may first communicate with the patient (e.g., in a chat window, application, or user interface displayed on the patient's user device). The chatbot module 124 may ask, “Hey, would you like to take a break and do a quick breathing exercise with me?” The chatbot module 124 may be able to evaluate the patient's response and continue to provide guidance or address the patient's desires. For example, the chatbot module 124 may further ask, “If you′d like to speak to one of our specialists, at any time please ask for assistance. But in the meantime, let's go through a somatic meditation checklist.” Depending on the patient's communications and/or the outcome of the checklist, the patient interaction can be escalated to a trained telemedicine practitioner (e.g., a proctor) as needed or even to an ambulance being dispatched to the patient's location.

In some embodiments, the telehealth proctoring platform 100 can include an intervention system or module 126. In some embodiments, the intervention module 126 can be configured to automatically implement various interventions based on a patient's health information and condition. In some embodiments, the intervention module 126 can be configured to present to a telemedicine practitioner a report that indicates possible ailments/conditions that a patient may have and the input data and analysis that was used in making that determination. In some embodiments, the intervention module 126 can be configured to present to a telemedicine practitioner (e.g., via a graphical user interface) a patient's health information and/or different options for implementing various interventions. In other words, the intervention module 126 may provide an evaluation and decision-making dashboard to a proctor. For example, a recovered drug addict may be wearing health sensors 110 (e.g., an Apple Watch, FitBit, or other health tracker) that gives a worrying indication. Through the intervention module 126, a proctor may be able to access a data panel on the patient that can include, for example, biomarkers such as blood oxygen levels, a graph of heart rate, a video feed from user device (e.g., smartphone) cameras, a microphone feed, an ability to speak out of the speakers, a three dimensional model of the patient moving around the room using IMU data, along with estimated patient orientation (laying on ground, sitting), limb movement via smartwatch IMU, etc. The intervention module 126 may be able to automatically review this information and determine an appropriate intervention for the telehealth proctoring platform 100 to take. Alternatively, the proctor can review this information to make a judgment call (e.g., to disconnect from the patient, to stay on the line and monitor, to start engaging with the patient, to dispatch an ambulance, and so forth). In some embodiments, patients may authorize an automatic activation of sensors on their devices (e.g., user devices 108, health sensors 110, and/or health monitors 112) for medical practitioners or the telehealth platform to make rapid judgment calls or pull any needed data.

In some embodiments, the telehealth proctoring platform 100 may include a motivation system or module 128. The motivation module 128 may track the various motivational programs that can improve patient adherence to a treatment plan or regimen, the motivational programs that are assigned to each patient, the proctor administering a particular motivational program for a patient, and so forth. In some embodiments, the motivation module 128 may handle the assignment of a motivational program to a patient. For example, a patient may voluntarily opt-in to or sign up for one or more motivational programs, or a proctor or clinician may assign one or more motivational programs to the patient, through a user interface option (e.g., interfaces 120) provided by the telehealth proctoring platform. That assignment may be performed by the motivation module 128. In some embodiments, the motivation module 128 may provide digital tools for patients to help remind them and keep them on track with the regimen. In some embodiments, the motivation module 128 may provide tools to a proctor for administering a motivational program, such as for generating artifacts (e.g., certificates to provide a patient for completing one or more portions of the health improvement regimen) or reports of patient compliance to be sent out to their healthcare provider.

Thus, it can be understood that the telehealth proctoring platform 100 described herein can offer many advantages. For example, the patients 102 may not need to travel for periodic check-ins. In some embodiments, the patients 102 can speak with proctors 104, clinicians 106, or other medical professionals (e.g., physician, nutritionist, health coach, and/or the like) on demand or with short notice (for example, immediately, a same-day appointment, an appointment within a few days, and so forth). In some embodiments, the telehealth proctoring platform 100 can be used to enable proctors 104, clinicians 106, or other medical professionals to provide guidance or instructions to a patient, such as instructions for performing a diagnostic test. In some embodiments, the patients 102 may be able to conduct some forms of testing at home under proctored supervision, such as checking blood glucose levels or for the presence of COVID-19.

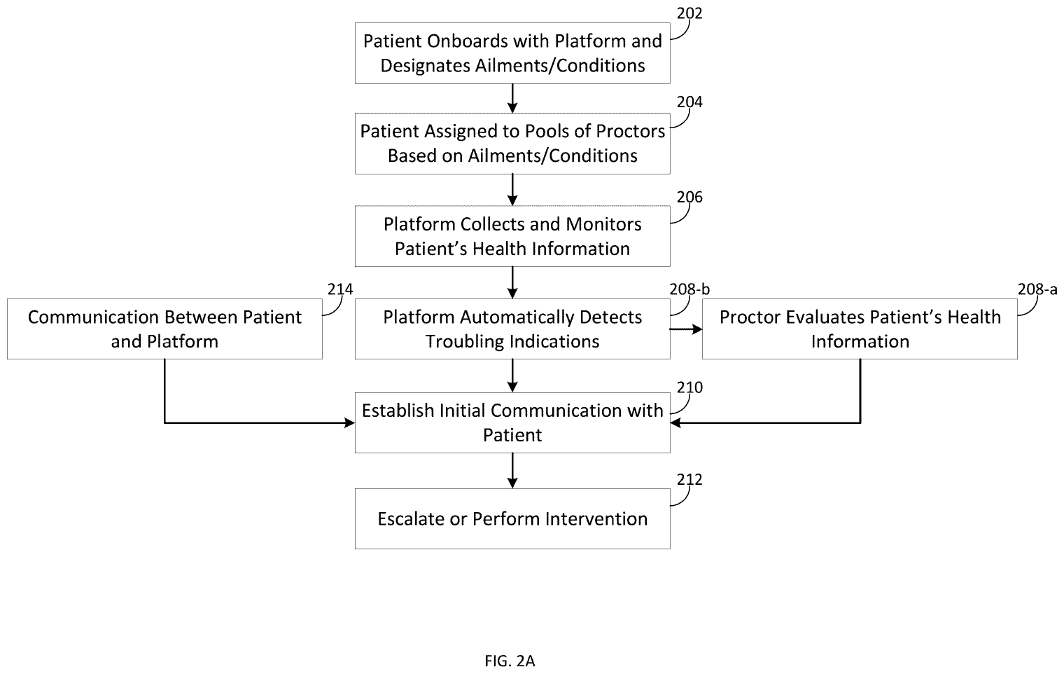

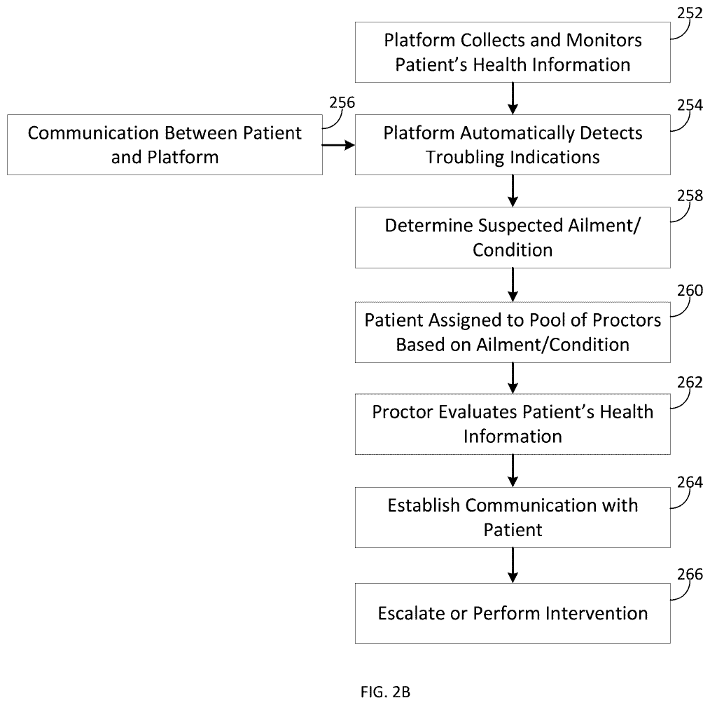

FIGS. 2A-2B illustrate block diagrams of example methods that can be used to provide faster and more specific monitoring, detection, and intervention for users of a telehealth proctoring platform, in accordance with embodiments disclosed herein. These methods can be implemented, for example, using one or more components of the system shown in FIG. 1.

As previously mentioned, traditional medicine is poorly set up to provide immediate care to patients—especially care that will be responsive to the patient's exact needs or medical condition. Patients desiring a consultation will have to schedule appointments on future dates and at physically distant locations. Even emergency services like paramedics or emergency rooms require travel time, going to a physical hospital, and sometimes long waits. This poses a problem for patients with medical issues that require time-sensitive interventions.

Telehealth is uniquely positioned to address these issues in a way that would be impossible for traditional medical paradigms. For example, a telehealth proctoring platform can be configured to be always and immediately accessible to its users to provide guidance and consultation on a specific medical issue. As another example, due to the increasing prevalence of user tracking devices or health tracking devices (e.g., health sensors or health monitors) and wearable technology (e.g., smart watches with health sensors), health data for a patient can be collected and transmitted to a telehealth proctoring platform (e.g., continuously, asynchronously, etc.). The patient's health data can be remotely monitored by the telehealth proctoring platform in real-time, such as by an artificial intelligence algorithm and/or a proctor, in order to detect abnormalities or the occurrence of an ailment or condition—perhaps even before the patient is aware. The telehealth proctoring platform can immediately establish communication and provide an intervention based on the specific medical issue.

FIG. 2A is directed towards an approach where the patient is first assigned to various pools of proctors trained to handle specific ailments/conditions. This association can then be used to determine the kind of patient/user status data needed, how that data should be collected/transmitted, the algorithms that will have to be executed on that data, and so forth—thereby increasing efficiency and scalability (e.g., reducing the amount of data/transmissions/processing that would be required).

At block 202, a patient (e.g., a user) may sign up and onboard with the telehealth proctoring platform. The telehealth proctoring platform may determine certain ailments or medical conditions that the patient suffers from, that the patient is predisposed to developing or at risk of developing, and so forth. For example, the patient may designate those ailments or medical conditions in their profile or in a questionnaire, the platform may automatically determine those ailments or medical conditions by reviewing the patient's health history/records and/or the patient's answers to a questionnaire, a referring physician may have indicated those ailments or medical conditions, and so forth. In some embodiments, the telehealth proctoring platform may save information about the patient, such as the ailments/conditions associated with the patient, in a database. The database may also contain any useful information associated with those ailments/conditions or the patient, such as certain triggers (e.g., large crowds or claustrophobic areas give this patient anxiety) or patient idiosyncrasies (e.g., this patient typically fasts on Fridays which can be associated with, and explains, lower blood sugar on those days). The telehealth proctoring platform or a proctor may be able to take this information into account when monitoring a patient's health information or deciding on an intervention.

At block 204, the telehealth proctoring platform may assign the patient to one or more pools of proctors based on those ailments/conditions. There may be a pool of proctors for each specific ailment/condition, with those proctors having experience and training for that specific ailment/condition. Patients with multiple ailments/conditions can be assigned to multiple pools. For example, assume a patient is associated with both hypertension and anxiety attacks (categorized under two separate ailments/conditions within the platform). The patient may be assigned to a first pool of proctors who are trained to deal with hypertension and assigned to a second pool of proctors who are trained to deal with anxiety attacks. A proctor could be part of both the first pool and the second pool (e.g., a proctor trained in both hypertension and anxiety attacks). In some embodiments, the telehealth proctoring platform may save information about these assignments and groupings into a database, such as a list of the proctors in each pool or associated with each ailment/condition, a list of pools each patient is assigned to, a list of patients assigned to each pool, and so forth.

At block 206, the telehealth proctoring platform may collect and monitor a patient's health information (e.g., patient or user status data, which can be collected in real-time). For example, a patient may have user tracking devices or health tracking devices, such as health sensors and/or health monitors, for collecting, tracking, and monitoring a patient's real-time data (e.g., health information, bioindicators, physiological data, vitals, activities etc.). Examples of such devices include wearable technology, such as smart rings, smart watches, Fitbit trackers, and so forth.

These health tracking devices may transmit the patient's health information to the telehealth proctoring platform. In some embodiments, the health tracking devices may be able to directly send the data to the telehealth proctoring platform. In some embodiments, the health tracking devices may indirectly send the data to the telehealth proctoring platform, such as by first providing the data to an intermediary device (e.g., a user device, a router or gateway, an edge server, a smart object, and so forth) to transmit to the telehealth proctoring platform. In some embodiments, the data may be transmitted continuously to the telehealth proctoring platform (e.g., in a real-time, near real-time, or substantially real-time stream). In some embodiments, the data may be transmitted to the telehealth proctoring platform periodically or at regular or continuous intervals. In some embodiments, the data may not be transmitted to the telehealth proctoring platform at regular intervals. In some embodiments, the data may be sent asynchronously to the telehealth proctoring platform (e.g., the data is transmitted intermittently rather than in a steady stream, with timing information encoded in the data).

In some embodiments, some or all the data may be pooled and batched for transmission to the telehealth proctoring platform, thereby reducing the load and energy draw on the health tracking devices, the volume of transmissions, and bandwidth usage. For example, instead of a smart watch (or a user's device) continuously transmitting data to the telehealth proctoring platform, the smart watch may save up data for the most recent hour to be transmitted on the hour (or a different time interval can be used). Or the smart watch could save up data to be transmitted based on an event, such as once the smart watch is plugged in for charging, when the smart watch (or the user's device) is connected to a Wi-Fi network, and so forth.

In some embodiments, the manner of transmission and the kind of data transmitted in that manner can be based on the ailments/conditions that have been associated with a patient. For example, for ailments/conditions that may involve or require time-sensitive intervention (e.g., the patient could be having a heart attack), the data predictive of or associated with those ailments/conditions (e.g., heart rate or ECG/EKG signal) may be transmitted continuously or at shorter intervals. For ailments/conditions where intervention is not as immediately time sensitive, such as diabetes-related hyperglycemia or hypoglycemia, data predictive of or associated with those ailments/conditions (e.g., blood glucose levels over time) can be transmitted at longer intervals or based on an event. And for ailments/conditions where intervention is not very time sensitive, such as chronic sleep deprivation, data predictive of or associated with those ailments/conditions (e.g., sleep patterns) can be transmitted at even longer intervals or based on events that occur less frequently.

In some embodiments, the telehealth proctoring platform may collect the patient's health information it receives over time and save it to a database and/or the patient's health record. Thus, the telehealth proctoring platform may have a more complete data record that allows the observation of trends in the data that form over time in addition to the data or measurements that correspond to a single point in time or a small window in time.

The telehealth proctoring platform may monitor a patient's health information in a variety of ways. For example, at block 208-b, the telehealth proctoring platform may be able to automatically detect troubling indications or anomalies in the patient's health information. More specifically, the telehealth proctoring platform may use one or more artificial intelligence algorithms to monitor the patient's health information to perform various tasks, including making detections, performing classifications, generating predictions, and learning patterns from the data. Furthermore, the telehealth proctoring platform may also be able to use one or more artificial intelligence algorithms that leverage those tasks and information to automatically make determinations including diagnosing the patient's condition or making decisions about intervention.

There may be different kinds of algorithms used, and an algorithm may be tailored to a particular type of data or measurement. For instance, there may be algorithms that establish a baseline level or range (e.g., based on population data or the patient's historical data) that would be considered normal. Measurements that deviate too far from the baseline level or range may be considered abnormal. Or measurements that deviate too far from the baseline level or range for too long of a time period may be considered abnormal. Alternatively, there may be algorithms that establish a cutoff level or a range that would be considered abnormal (based on population data or the patient's historical data) and measurements above/below that level, or within the range, would be flagged for abnormality. As a specific example, for a patient with hypertension, the patient's blood pressure may be monitored to detect spikes in the blood pressure (e.g., an abnormally high blood pressure) that deviate against a baseline or are above a cutoff level. This determination can then be one of the factors used by another algorithm in determining an intervention to take (e.g., connect the patient to a proctor in a virtual proctoring session, during which the patient can perform a somatic meditation while the proctor assesses if the patient needs to go to the hospital for a heart attack risk).

As another example, there may be algorithms that employ machine learning techniques and approaches, including supervised learning, unsupervised learning, reinforcement learning, deep learning, and feature learning/reduction, and so forth. For instance, machine learning models can be trained to generate predictions or perform classifications based on various types of data or measurements available in the patient's health information. As a specific example, a machine learning model could be trained on electrocardiogram (ECG) readings to recognize the distinct patterns that are associated with (or are predictive of) different types of heart attacks. It could be used on a patient's ECG data to diagnose and classify heart attacks; when it detects the onset of a heart attack, an ambulance could be called to the patient's location and the paramedics can informed of the type of heart attack in order to make the proper medical response. Or, the model may be used on the ECG data of patients with chest pain in order to classify the patients for varying levels of heart attack risk (e.g., low, intermediate or high risk), and the appropriate intervention could be chosen based on the risk level. Additional inputs beyond ECG data could be used as well if it improves predictiveness, such as patient information that includes existing pain/symptoms, age, risk factors (e.g., smoking, diabetes, high cholesterol), patient and family heart attack history, blood levels of various biomarkers (e.g., proteins like troponin) if the data can be obtained from the patient's health tracking devices, and so forth.

In many cases, after the telehealth proctoring platform has detected troubling indications or anomalies in the patient's health information, sufficiently diagnosed the patient's condition, and/or determined an intervention to take, communication with the patient may be established at block 210. There may be various ways to establish communication with the patient. In some embodiments, the communication approach may depend on the patient's ailment/condition and/or the intervention.

For instance, for some interventions, the telehealth proctoring platform may establish one-way communication, such as by messaging the patient to let the patient know that an ambulance is being sent to their location. For some other interventions, it may be desirable to establish communication between the patient and a proctor right away. The telehealth proctoring platform could establish a virtual proctoring session (e.g., a live video conference) or the proctor could message or call the patient (e.g., the platform may give the proctor the patient's contact details). In some embodiments, the proctor assigned to the patient may be a proctor that is selected from the pool of proctors assigned to the patient based on the ailment/condition (designated at block 204). For instance, the patient may have been assigned to a pool of proctors that anxiety attacks.

For yet other interventions, it may be desirable to provide minimal intervention with optional escalation of care. For example, the telehealth proctoring platform can employ a system, such as an artificial intelligence system like a chatbot utilizing aspects of deep learning and natural language processing, configured to provide initial communication with the patient and maintain a conversation with the patient in natural language and simulating the way a human would behave as a conversational partner. As a specific example, if the patient is determined to be having an anxiety attack, the system could ask the patient, “Hey, would you like to take a break and do a quick breathing exercise with me?” The system could then walk the patient through the breathing exercise. The system can then further ask, “If you′d like to speak to one of our specialists, at any time please ask for assistance. But in the meantime, let's go through a somatic meditation checklist.” The system could then walk the patient through the checklist. Depending on the outcome of the checklist, at block 212, the patient interaction can be escalated to a trained telemedicine specialist as needed or even to an ambulance being dispatched.

Thus, due to the increasing prevalence of wearable technology, the platform can use data such as bioindicators from health tracker sensors, GPS, or IMUs to detect these time-sensitive ailments perhaps even before the patient is aware of them. For example, a patient with hypertension whose blood pressure spiked may be automatically connected with a proctor who has them perform a somatic meditation while also assessing if they need to go to the hospital for a heart attack risk. As another example, a patient with anxiety attacks might have elevated BPM and hyperoxygenation of the blood detected along with a GPS location indicating they are near one of their triggers (e.g., large crowds, a workplace, etc.), and so may be contacted before the patient realizes they are about to have an attack to help guide them through it or even prevent it.

In addition to the use of algorithms for automated monitoring, a patient's health information could also be monitored by a proctor. For example, after the platform detects troubling indications, the platform may compile into a report that includes any of the patient information, health information, patient/user status data, etc., that was relevant up to this point in detecting the troubling indications. This report may be reviewed at block 208-a by a proctor, who could evaluate the patient's health information and make a determination regarding the meaning of the detected troubling indication, the appropriate intervention to take, and so forth. In some embodiments, the report may be sent to a proctor from the assigned pool (e.g., at block 204) or it may be flagged for review by the proctors of the assigned pool, and a proctor from the assigned pool (e.g., a proctor that currently has availability) may look at it. Afterwards, the proctor may establish contact with the patient (e.g., at block 210) in order to perform the appropriate intervention.

As a very specific example, a patient may be recovered drug addict wearing an Apple Watch, FitBit, or other health tracker that provides data associated with a worrying indication about potential relapse. In response, a telemedicine practitioner from the assigned pool may be notified (e.g., a proctor trained in drug rehabilitation). In some cases, without needing the patient's permission, the proctor may be able to pull up a data panel on the patient that can include, for example, biomarkers such as blood oxygen levels, a graph of heart rate, a video feed from either/both device (e.g., smartphone) cameras, a microphone feed, an ability to speak out of the speakers, a three dimensional model of the patient moving around the room using IMU data, along with estimated patient orientation (laying on ground, sitting), limb movement via smartwatch IMU, etc. Based on this information, the proctor or platform could then make a judgment call to disconnect, to stay on the line and monitor, to start engaging with the patient (e.g., in a video call or a voice call or other communication), or to dispatch an ambulance. Some patients may even authorize an automatic activation of sensors on their devices for medical practitioners or the telehealth platform to make rapid judgment calls.

Furthermore, a patient may be able to take initiative and make inquiries with the telehealth proctoring platform. And after a patient has already designated an ailment/condition, if the patient communicates with the platform at block 214 (e.g., via an AI chatbot) about the condition and it is determined that the patient should communicate directly with a proctor (e.g., in a virtual proctoring session), the proctor may be selected from the pool assigned to the patient based on availability. That proctor will be knowledgeable about the condition and will be well suited to discuss the matter with the patient. For example, a patient may be susceptible to anxiety attacks and they believe they are about to have an anxiety attack. The patient may communicate this to the platform at block 214, which may determine that the patient should immediately speak with a proctor to prevent the anxiety attack from occurring. Then at block 210, a communication (e.g., a virtual proctoring session) between the proctor and the patient can then be immediately set up, with the proctor selected from the pool of proctors trained to mitigate anxiety attacks.

FIG. 2B is directed towards an approach where patient/user status data (e.g., patient health information) is collected and monitored. Based on the monitored data and/or additional patient communication/data, the patient may be suspected of having a specific ailment or condition. The patient can be assigned on-the-fly to a pool of proctors to handle that ailment or condition. A proctor from this pool of proctors can be selected to review the patient's situation to help confirm the ailment/condition, determine interventions to take, and/or communicate with the patient.

This approach may cast a wider net during monitoring for ailments/conditions that can be detected. At the same time, the approach would also increase efficiency and scalability. It uses systems/algorithms (which are better suited than humans for processing vast quantities of patient status data or patient health information) to make a first pass at detecting a suspected ailment/condition and compile a report that can be flagged for proctor review (who are better at on-the-fly, flexible decision making). The proctor can be specifically selected from a pool of proctors trained in the suspected ailment/condition, which improves decision making quality and scalability.

At block 252, the telehealth proctoring platform may collect and monitor a patient's health information (e.g., patient or user status data, which can be collected in real-time). Details for this step may be similar to those discussed for block 206 of FIG. 2A. The various types of data and measurements collected may depend on the kinds of user/health tracking devices available to the patient.

At block 254, the telehealth proctoring platform may automatically detect troubling indications within the patient's health information by applying different sets of algorithms on the information. Details for this step may be similar to those discussed for block 208-b of FIG. 2A. However, the algorithms that can be applied may depend on the various types of data and measurements of the patient that are available, which in turn may depend on the kinds of user/health tracking devices available to the patient. In general, the more kinds of user/health tracking devices available to the patient and transmitting to the telehealth proctoring platform, the greater the number of troubling indications that can be detected within the patient's health information.

In some cases, at block 256, there may be a communication between the patient and the telehealth proctoring platform, which may trigger the platform to perform the detections at block 254 or may supply additional patient information that can be used to perform the detections. Furthermore, the telehealth proctoring platform may inquire for additional patient information for performing the detections. For example, the patient may communicate with the telehealth proctoring platform indicating that the patient has chest pains. Thus, the platform has a better idea of what type of data and measurements to look at, and armed with this additional information, can run algorithms at block 254 to detect any troubling indications or abnormalities in the patient's data that could be related to the patient's chest pains (e.g., detect abnormalities in the ECG for evaluating the possibility of a heart attack).

At block 258, based on any troubling indications detected in the patient's data and any patient information known to the platform, the telehealth proctoring platform may determine one or more suspected ailments or conditions that are most likely associated with those troubling indications. For instance, the telehealth proctoring platform may have determined that there was an abnormal spike in blood pressure, that the patient did not recently consume any drugs or medication (e.g., caffeine), and that there are low chances of a heart attack occurring, and so forth. Based on this information, the platform may determine that the most likely condition is hypertension.

At block 260, the telehealth proctoring platform may assign the patient to one or pools of proctors trained to handle the suspected ailments/conditions. Details for this step may be similar to those discussed for block 204 of FIG. 2A. For example, after determining that the most likely condition the patient is suffering from is hypertension, the platform may assign the patient to a pool of proctors that are trained to handle hypertension.

In some cases, at block 262, the platform may compile into a report all the patient information, health information, patient/user status data, etc., that was relevant up to this point in making determinations on troubling indications and the suspected ailments/conditions. The report may also indicate the detected troubling indications and the suspected ailments/conditions. This report may get flagged for review by the proctors of the assigned pool, and a proctor from the assigned pool (e.g., a proctor that currently has availability) may pick it up to evaluate the patient's health information and make a determination regarding the patient's ailment/condition, the appropriate intervention to take, and so forth.

At block 264, the platform may establish a communication with the patient. Details for this step may be similar to those discussed for block 210 of FIG. 2A. For instance, an AI chatbot or the proctor may reach out to the patient (e.g., in a virtual proctoring session) to deliver the diagnosis or inform them of the intervention.

At block 266, the intervention can be performed (e.g., the proctor or chatbot walks the patient through a breathing exercise) or the communication/interaction can be escalated. Details for this step may be similar to those discussed for block 212 of FIG. 2A. For instance, the interaction may be forwarded to a trained telemedicine specialist, an ambulance can be dispatched, and so forth.

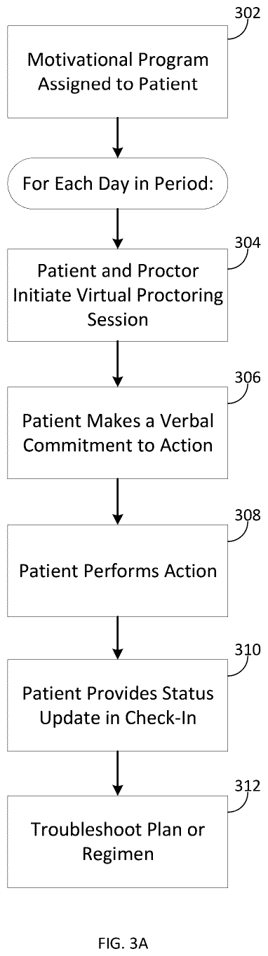

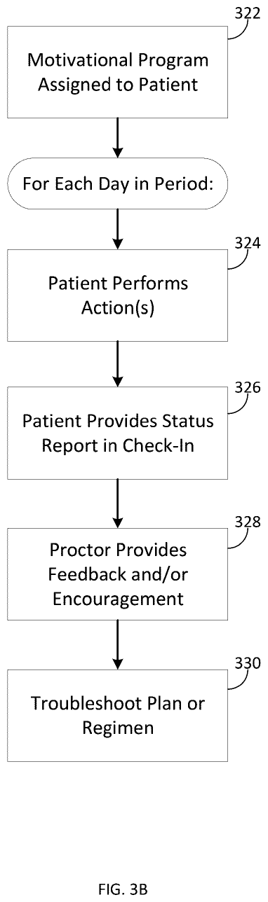

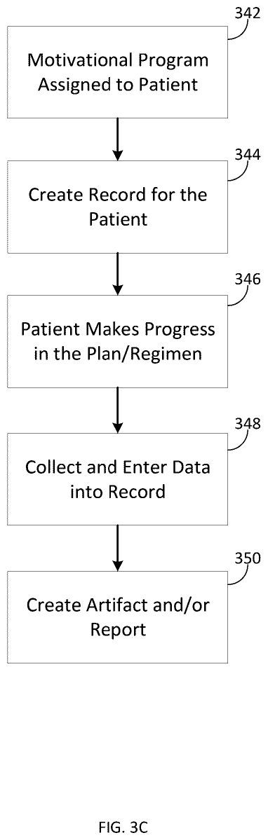

FIGS. 3A-3C illustrate block diagrams of methods associated with different example motivational programs that can be assigned to a patient enrolled in a medical treatment plan managed over a telehealth proctoring platform. The method can be implemented, for example, using one or more components of the system shown in FIG. 1.

Patients are often enrolled a medical treatment plan or health improvement regimen (e.g., a weight loss program), which can include drug abstinence, medicine or prescription schedules, diet, exercise, and/or any other health improvement or lifestyle change. Patient adherence to the health improvement regimen can be a leading indicator of whether the health improvement regimen will be successful in improving patient health. In order for long term adherence to a habit to occur (e.g., a patient's adherence to a health improvement regimen), the first 60-90 days are critical. In this time period of a health they patient skipped a day, broke the habit, or did not perform a task required under the regimen. Typically, these excuses fall apart under scrutiny. However, without the scrutiny, patients can convince themselves that their excuses are legitimate.

In some embodiments, patients may be able to access the telehealth proctoring platform disclosed herein (e.g., online, such as through a web-based application or an installed application on their device) while being enrolled in a medical treatment plan or health improvement regimen. For instance, the telehealth proctoring platform may be configured to allow a patient to routinely check in with a proctor in virtual proctoring sessions (e.g., an audio or video call that allows the proctor to communicate with the patient) to demonstrate compliance with the health improvement regimen.

In some embodiments, the telehealth proctoring platform may include a number of different motivational schemes or programs that can be assigned to a patient enrolled in a medical treatment plan or health improvement regimen. For instance, FIGS. 3A-3C depict three different motivational programs for exemplary purposes, and this is not meant to be an exhaustive list. Furthermore, steps and functionality described for one motivational program may be used with another motivational program or implemented in a varying order.

One or more motivational programs can be assigned to a patient directly through the telehealth proctoring platform. Assigning one of these motivational programs may improve patient adherence to the treatment plan or regimen; the motivational programs may be especially assigned to high-risk patients for whom adherence to a treatment plan is critical. There may be various ways that a motivational program can be assigned to a patient. For example, a patient may voluntarily opt-in to or sign up for the one or more motivational programs for undergoing self-improvement, habit changes, and/or other lifestyle changes. In some cases, the patient may be able to sign up by selecting a user interface option provided by the telehealth proctoring platform. Alternatively, a proctor or clinician may assign one or more motivational programs to the patient (especially a high-risk patient for whom adherence to a treatment plan is critical).

In some embodiments, the telemedicine practitioners (e.g., proctors) tasked with managing and administering a motivational program may be pulled from a group of idle proctors who may typically administer other telehealth services on the platform (e.g., supervision of COVID19, flu, UTI, HPV testing, etc.). In other words, there may be many proctors that simultaneously use the telehealth proctoring platform to perform many different tasks, but there can be a number of proctors who are idle at any point in time. These spare proctors can be used to serve as a point of accountability and improve patient adherence. Furthermore, using spare proctors in this manner can help balance resource utilization curves and improves the efficiency and scalability of the telehealth proctoring platform.

And importantly, these motivational programs may allow for troubleshooting of the medical treatment plan applied to a patient. A proctor may be able to utilize the data provided by the patient in connection with a motivational program to uncover the reasons behind the patient's poor adherence to the treatment plan. The proctor can evaluate those reasons and make modifications to the medical treatment plan so that the treatment plan is better suited for the patient, thereby improving patient adherence to the treatment plan.

In some embodiments, the telehealth proctoring platform or system can group a plurality of patients together (e.g., a group of 5 patients). The patients can be assigned the same health improvement regimen. The patients can also be assigned the same motivational program. In some embodiments, a group of patients may check in to the proctor together as a “class” to build community. Patients within a group may benefit from hearing how others dealt with challenges and may be able to provide helpful suggestions to others. This may serve to reinforce commitment to the plan and to motivate follow-through. Thus, the plurality of patients in the group can provide support to each other and each patient may receive accountability from other patients experiencing similar challenges. The plurality of patients can provide suggestions for how to deal with challenges and/or provide support to each other.

In some embodiments, aspects of a health improvement regimen and/or motivational program (e.g., certain steps) can be linked with one or more devices, events, and/or activities in the patient's day. For example, an alarm clock going off may trigger a timer indicating what time the patient's check in is at (e.g., 30 min., 1 hr., or other time selectable by the patient). The timer can provide a countdown until the patient's first proctor meeting for the day. In some embodiments, aspects of a health improvement regimen and/or motivational program can be tied to the patient's location or the location of a device of the patient. For example, the telehealth proctoring platform or system may be able to access a location of a device of the patient. When the location of the device indicates the patient is home from work, school, or any other task, the system can trigger the timer. As a more specific example, a GPS reading that the user pulled into their driveway after work may trigger a timer countdown until the patient's proctor check-in, etc.

FIG. 3A depicts a motivational program involving the use of verbal commitment.

At block 302, the motivational program is first assigned to the patient (e.g., through the telehealth proctoring platform). For example, the patient may have voluntarily opted-in or signed up for the motivational program, or a proctor or clinician may have assigned the motivational program to the patient.

In some embodiments, this motivational program may be used over a certain time period. For example, the motivational program (e.g., blocks 304, 306, 308, 310, and/or 312) may be adhered to each day during the first 90 days of habit building.

Thus, for each day in this period, at block 304, a virtual proctoring session may be initiated between the patient and a proctor. In some cases, the patient and the proctor may meet early in the day (e.g., in the morning, before school or work or within a few hours of waking up).

At block 306, during the virtual proctoring session, the patient may make a verbal commitment to the practitioner to adhere to some aspect of the medical treatment plan. For example, the medical treatment plan may involve the performance of certain steps or actions (or even inaction—e.g., do not eat any food before noon). Thus, the patient may make a verbal commitment about: (a) what action will be performed or prevented; (b) when the action will be performed, and/or; (c) how the action will be performed. This act of making a plan and expressing the commitment externalizes the commitment and places it concretely into the patient's mental calendar. This makes success (defined by following through with the expressed plan) more likely.

At block 308, the virtual proctoring session will be over. The patient will go about their day. In the course of performing their day-to-day activities, the patient may perform the action that they verbally committed to during the virtual proctoring session. Or, alternatively, the patient may end up failing to perform the action for whatever reason (e.g., the patient eats food before noon because the patient's afternoon suddenly becomes completely scheduled).

At block 310, the patient will check-in with the proctor and provide status updates. For example, this check-in can occur in-app, by phone, or by message, depending on the most-effective check-in method and/or individual patient preferences. The patient will indicate whether they performed or failed to perform the action that they verbally committed to.

At block 312, the proctor may help troubleshoot obstacles that prevented full completion of the plan (e.g., come up with a solution that prevents the patient's entire afternoon from becoming scheduled). This may also be an opportunity for the patient and proctor to analyze the plan/regimen and make revisions or modifications if needed (e.g., permit the patient to eat before noon but restrict the patient from eating after 5 pm). In some embodiments, plan revisions may require that the patient discuss it with their clinician that prescribed the plan.

Afterwards, the blocks 304, 306, 308, 310, and/or 312 may be repeated each day over the specified time period (e.g., first 90 days).

FIG. 3B depicts a motivational program involving the use of congratulation and encouragement.

At block 322, the motivational program is first assigned to the patient (e.g., through the telehealth proctoring platform). For example, the patient may have voluntarily opted-in or signed up for the motivational program, or a proctor or clinician may have assigned the motivational program to the patient.

In some embodiments, this motivational program may be used over a certain time period. For example, the motivational program (e.g., blocks 324, 326, 328, and/or 330) may be performed daily over a number of days.

Thus, for each day in this period, at block 324, the patient may perform and complete the daily actions or tasks specified by the medical treatment plan/regimen. In some embodiments, visuals may be provided to the patient as a reminder of their progress. For example, a digital calendar with planned tasks for the day may be provided within the app or online. The digital calendar can identify or stylize completed tasks and uncompleted tasks. The tasks may be stylized such that completed tasks are easily recognized as such (e.g., strikethrough font), while uncompleted tasks are represented differently (e.g., bold, highlighted, or blinking, etc.). Marking tasks off the reminder list may not replace the need to check in with the proctor but could serve as an individual reminder about the status of tasks.

At block 326, the patient may check-in with a proctor and provide a status report. This check-in may occur via a video conference (e.g., in a virtual proctoring session), over messaging, over phone, etc. For example, the patient may report the completion of the day's tasks specified by the regimen and also provide any relevant information (e.g., it was hard to abstain today, I ate 660 calories, I took my full dose, I completed 5×5 80 lb squats, etc). The proctor may hear all the information provided by the patient and enter it in their file or report associated with the patient.

By sharing the accomplishment with someone who cares, a sense of community is provided to the patient and it further externalizes the motivation for the activity. The check-in also serves as a reminder/reinforcement to the patient that they were able to complete their plan for the day. This may improve confidence and healthy habit building while also providing peace of mind (e.g., avoiding the “did I already take that pill this morning?” scenario).

In some cases, if the end of the day (or some other scheduled trigger event) lapses and the patient has not checked in, the patient may be automatically connected with a proctor (e.g., the proctor calls or messages the patient). The patient may have to explain to the proctor why they did not adhere to the regimen that day or why they forgot to check-in with the proctor. Since excuses made to oneself are much easier to justify than excuses made to another person, even a stranger, this check-in may increase the mental burden of not sticking to the regimen and its activities and also increase the mental reward of sticking with it.

At block 328, the proctor may provide the patient with feedback and congratulate or encourage the patient for completing all their daily tasks specified by the regimen. In some embodiments, an animation representing “success” may be played and displayed to the patient as an enthusiastic congratulatory reward. The proctor may also congratulate and/or encourage the patient if the patient completed only part of their plan.

At block 330, if the patient completed only part of their plan or failed to perform any tasks specified in the plan, then the proctor may help the patient troubleshoot any obstacles that prevented full completion of the plan. In some embodiments, this may be an opportunity for the patient and the proctor to analyze the plan and make revisions if needed. In the case of medical adherence, plan revisions may require that the patient discuss with their clinician (e.g., that prescribed the plan/regimen). This may happen through the app (e.g., physician/physician's team receives a notification that the patient is not able to complete their assigned regimen and then makes a check-in call; schedules a call/video conference in the app or through the website; or schedules the patient for an in-office visit).

Afterwards, the blocks 324, 326, 328, and/or 330 may be repeated each day over the specified time period.

FIG. 3C depicts a motivational program involving the generation of records, artifacts, and/or reports.

At block 342, the motivational program is first assigned to the patient (e.g., through the telehealth proctoring platform). For example, the patient may have voluntarily opted-in or signed up for the motivational program, or a proctor or clinician may have assigned the motivational program to the patient.

At block 344, a record may be created for the patient within the telehealth proctoring platform/system. In some embodiments, data populating the record can only be input by a telemedicine employee or practitioner (e.g., a proctor and/or clinician). This setup makes falsifying data more difficult for the patient—the patient would have to verbally falsify data to another human, which is harder than justifying it to themselves, especially when the proctor is trained to ask a few pressing questions.

At block 346, the patient may make progress in the prescribed plan/regimen over time. For instance, the patient may perform daily a set of tasks or actions that are specified by the plan/regimen.

At block 348, data may be collected and entered into the record for the patient. The data that is collected and entered may be dependent on the individual patient's regimen. Examples include weight/body measurements, # of days of adherence, # of days since last habit break, % towards goal, etc. Data may be continually collected and entered into the record over time (e.g., as the patient continues to make progress in the prescribed plan/regimen). In some embodiments, a telemedicine employee or practitioner (e.g., a proctor and/or clinician) may have to collect and enter this data into the record for the patient. For instance, a proctor may have regular check-ins or virtual proctoring sessions with the patient, through which the proctor can determine patient measurements or behavior.

At block 350, artifacts and/or reports may be created within the telehealth proctoring platform or system. Artifacts created can be as simple as a certificate for completing one or more portions of the health improvement regimen or for the patient passing certain milestones, a visualization of success such as a graph, a physical object, a figurine, a trophy, and/or a graphic that can be shared via email, social media, or other communication method to illustrate progress to friends and family. An artifact creates a real-world tangible representation of the actions being taken by the patient, and it can serve both as accountability and motivation. Reports may capture the patient's progress and may be generated for the patient to provide to his or her physician/healthcare provider. In some embodiments, such reports may be provided to the physician directly upon request, at regular intervals, when a patient misses a check-in, and/or upon completion of the program.

### Computer Systems

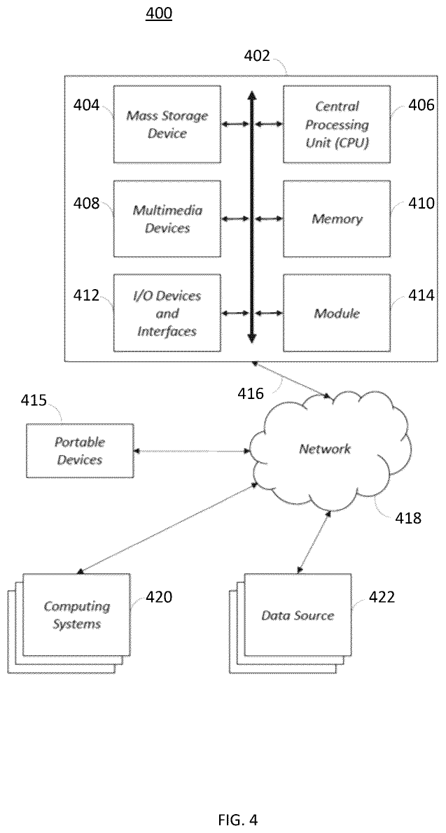

FIG. 4 is a block diagram depicting an embodiment of a computer hardware system configured to run software for implementing one or more embodiments of the telehealth proctoring platform and any systems, methods, and devices disclosed herein. The example computer system 402 is in communication with one or more computing systems 420 and/or one or more data sources 422 via one or more networks 418. While FIG. 4 illustrates an embodiment of a computing system 402, it is recognized that the functionality provided for in the components and modules of computer system 402 may be combined into fewer components and modules, or further separated into additional components and modules.

The computer system 402 can comprise a module 414 that carries out the functions, methods, acts, and/or processes described herein. The module 414 is executed on the computer system 402 by a central processing unit 406 discussed further below.

In general, the word “module,” as used herein, refers to logic embodied in hardware or firmware or to a collection of software instructions, having entry and exit points. Modules are written in a program language, such as JAVA, C or C++, PYPHON or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 402 includes one or more processing units (CPU) 406, which may comprise a microprocessor. The computer system 402 further includes a physical memory 410, such as random-access memory (RAM) for temporary storage of information, a read only memory (ROM) for permanent storage of information, and a mass storage device 404, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 402 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 402 includes one or more input/output (I/O) devices and interfaces 412, such as a keyboard, mouse, touch pad, and printer. The I/O devices and interfaces 412 can include one or more display devices, such as a monitor, which allows the visual presentation of data to a user. More particularly, a display device provides for the presentation of GUIs as application software data, and multi-media presentations, for example. The I/O devices and interfaces 412 can also provide a communications interface to various external devices. The computer system 402 may comprise one or more multi-media devices 408, such as speakers, video cards, graphics accelerators, and microphones, for example.

The computer system 402 may run on a variety of computing devices, such as a server, a Windows server, a Structure Query Language server, a Unix Server, a personal computer, a laptop computer, and so forth. In other embodiments, the computer system 402 may run on a cluster computer system, a mainframe computer system and/or other computing system suitable for controlling and/or communicating with large databases, performing high volume transaction processing, and generating reports from large databases. The computing system 402 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 402 illustrated in FIG. 4 is coupled to a network 418, such as a LAN, WAN, or the Internet via a communication link 416 (wired, wireless, or a combination thereof). Network 418 communicates with various computing devices and/or other electronic devices. Network 418 is communicating with one or more computing systems 420 and one or more data sources 422. The module 414 may access or may be accessed by computing systems 420 and/or data sources 422 through a web-enabled user access point. Connections may be a direct physical connection, a virtual connection, and other connection type. The web-enabled user access point may comprise a browser module that uses text, graphics, audio, video, and other media to present data and to allow interaction with data via the network 418.

Access to the module 414 of the computer system 402 by computing systems 420 and/or by data sources 422 may be through a web-enabled user access point such as the computing systems' 420 or data source's 422 personal computer, cellular phone, smartphone, laptop, tablet computer, e-reader device, audio player, or another device capable of connecting to the network 418. Such a device may have a browser module that is implemented as a module that uses text, graphics, audio, video, and other media to present data and to allow interaction with data via the network 418.

The output module may be implemented as a combination of an all-points addressable display such as a cathode ray tube (CRT), a liquid crystal display (LCD), a plasma display, or other types and/or combinations of displays. The output module may be implemented to communicate with input devices 412 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 402 may comprise a physical or logical connection established between a remote microprocessor and a mainframe host computer for the express purpose of uploading, downloading, or viewing interactive data and databases on-line in real time. The remote microprocessor may be operated by an entity operating the computer system 402, including the client server systems or the main server system, an/or may be operated by one or more of the data sources 422 and/or one or more of the computing systems 420. In some embodiments, terminal emulation software may be used on the microprocessor for participating in the micro-mainframe link.

In some embodiments, computing systems 420 who are internal to an entity operating the computer system 402 may access the module 414 internally as an application or process run by the CPU 406.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 402 may include one or more internal and/or external data sources (for example, data sources 422). In some embodiments, one or more of the data repositories and the data sources described above may be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 402 may also access one or more databases 422. The databases 422 may be stored in a database or data repository. The computer system 402 may access the one or more databases 422 through a network 418 or may directly access the database or data repository through I/O devices and interfaces 412. The data repository storing the one or more databases 422 may reside within the computer system 402.

### Additional Embodiments

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

We claim:

1. A computer-implemented method for enabling a remote intervention of a user, the method comprising:
receiving, from a user device, a designation of a condition by a user;
assigning, based on the designation, the user to a pool of supervising users associated with the condition;
receiving, from a user tracking device, user status data;
establishing a baseline from the user status data;
detecting, by applying an algorithm associated with the condition to the user status data, an indication in the user status data that deviates from the baseline;
determining a remote intervention to be performed based on the condition and the detected indication;
selecting a supervising user from the pool of supervising users associated with the condition, wherein the supervising user is selected based on availability; and
establishing a communication between the supervising user and the user via the user device, wherein the communication is directed to performing the remote intervention.

2. The computer-implemented method of claim 1, wherein the condition is a medical condition.

3. The computer-implemented method of claim 1, wherein the user tracking device is a type of wearable technology.

4. The computer-implemented method of claim 1, wherein the real-time user status data comprises physiological data about the user.

5. The computer-implemented method of claim 1, wherein the algorithm is a machine learning model.

6. The computer-implemented method of claim 1, wherein the communication is a live video stream between the supervising user and the user.

7. The computer-implemented method of claim 1, wherein the supervising user is a medical professional.

8. A non-transient computer readable medium containing program instructions for causing a computer to perform a method for enabling a remote intervention of a user, the method comprising:
receiving, from a user device, a designation of a condition by a user;
assigning, based on the designation, the user to a pool of supervising users associated with the condition;
receiving, from a user tracking device, user status data;
establishing a baseline from the user status data;
detecting, by applying an algorithm associated with the condition to the user status data, an indication in the user status data that deviates from the baseline;
determining a remote intervention to be performed based on the condition and the detected indication;
selecting a supervising user from the pool of supervising users associated with the condition, wherein the supervising user is selected based on availability; and
establishing a communication between the supervising user and the user via the user device, wherein the communication is directed to performing the remote intervention.

9. The non-transient computer readable medium of claim 8, wherein the condition is a medical condition.

10. The non-transient computer readable medium of claim 8, wherein the user tracking device is a type of wearable technology.

11. The non-transient computer readable medium of claim 8, wherein the real-time user status data comprises physiological data about the user.

12. The non-transient computer readable medium of claim 8, wherein the algorithm is a machine learning model.

13. The non-transient computer readable medium of claim 8, wherein the communication is a live video stream between the supervising user and the user.

14. The non-transient computer readable medium of claim 8, wherein the supervising user is a medical professional.
